(19) United States
(12) Patent Application Publication
(10) Pub. No.: US 2011/0030688 A1
(43) Pub. Date: Feb. 10, 2011

(54) Breathing System

(75) Inventor: George Harold Meakin, Manchester (GB)

Correspondence Address: BAKER & DANIELS LLP, 300 NORTH MERIDIAN STREET, SUITE 2700, INDIANAPOLIS, IN, 46204, US

(73) Assignee: THE UNIVERSITY OF MANCHESTER, Manchester (GB)

(21) Appl. No.: 12/937,005

(22) PCT Filed: Apr. 14, 2009
(86) PCT No.: PCT/GB2009/000971

§ 371 Date: Oct. 8, 2010

(30) Foreign Application Priority Data
Apr. 11, 2008 (GB) 0806648.2

(51) Int. Cl. A61M 16/08

(52) U.S. Cl. 128/205.13

(57) ABSTRACT

Provided is a breathing system comprising a flexible reservoir bag (6) having bag walls and provided with an exhaled gas inlet and a gas outlet (7). The bag defines a passageway for flow of gas in a first direction. The gas outlet of the reservoir bag is in communication with a collecting tube (8) which defines a passageway for flow of gas in a second direction and in use is connectable to a gas collector. At least part of the walls of the bag extend beyond the sides of the collecting tube, and the first and second directions of gas flow are substantially parallel to, and laterally offset from, one another. Additionally, the collecting tube is attached to an outer surface of the reservoir bag.

The present invention relates to breathing systems which can be used to deliver gases to a patient and to remove gases exhaled by the patient, in particular anaesthetic gases. The patient may be a human being receiving treatment from medical staff or an animal receiving treatment from veterinary staff.

In one known system, generally described as the Jackson Rees T-piece system or Mapleson F system, a mask covers a patient's nose and mouth. The mask is connected via a conduit to one limb of a T-junction, a second limb of which receives a fresh gas supply and a third limb of which is connected to a reservoir tube leading to an open-tailed reservoir bag. The bag expands as the patient exhales and then contracts as the patient inhales. The bag therefore provides medical staff with a method of monitoring the breathing patterns of the patient. The bag also allows staff to ventilate a patient if breathing difficulties arise by partially occluding the gas outlet of the bag while simultaneously squeezing it to propel gas into the patient's lungs. This operation is normally achieved with one hand only leaving the other hand free to manage the patient's airway. Two-hand operation is considered to be an advantage of the Jackson Rees T-piece compared with valved systems which require manual ventilation to be interrupted in order to adjust the release pressure of the valve.

One of the major drawbacks of the Jackson Rees system is that waste anaesthetic gases are discharged from the open tail of the reservoir bag into the operating theatre atmosphere where they constitute a hazard to the health of operating theatre staff and their families. Epidemiological studies carried out since the 1960s suggest that chronic exposure to these gases can lead to an increased risk of spontaneous abortion, stillbirth, congenital abnormalities, infertility, cancer, liver and renal disease. Accordingly, “Control of substances hazardous to health” (COSHH—a UK government code of practice) states that employers must protect employees from possible harmful effects of anaesthetic gases and vapours by the installation of effective scavenging equipment.

In the past, attempts have been made to devise scavenging systems to collect waste anaesthetic gases from the Jackson Rees T-piece, but these have all suffered from disadvantages. In most of these systems, the exhaled gases are fed into a scavenging system through a tube attached to the open tail of the reservoir bag. This leads to the system becoming somewhat unwieldy and increases the risk of exposing the airway to excessively high pressures by kinking either the collecting tube or the open tailed bag. It may also increase the risk of disconnection of the system.

One approach proposed in the prior art is to provide a breathing system in which a tube linked to a scavenging system is removably attached to the open tail of a reservoir bag, by means of a bridging tube. It is suggested that a breathing system of this sort may readily be converted to one which does not include a scavenging system in the event that occlusion occurs.

It is an object of the present invention to provide a breathing system which obviates or mitigates some or all of the problems outlined above.

According to a first aspect of the invention there is provided a breathing system comprising a flexible reservoir bag having bag walls and provided with an exhaled gas inlet and a gas outlet, the bag defining a passageway for flow of gas in a first direction, and the gas outlet of the reservoir bag being in communication with a collecting tube which defines a passageway for flow of gas in a second direction and in use is connectable to a gas collector, wherein at least part of the walls of the bag extend beyond the sides of the collecting tube, and the first and second directions of gas flow are substantially parallel to, and laterally offset from, one another, and wherein the collecting tube is attached to an outer surface of the reservoir bag.

During normal use the first direction of flow of gas will be from the exhaled gas inlet through the bag to the gas outlet, and the second direction of flow of gas will be away from the gas outlet and through the collecting tube. The gas outlet represents the route by which waste gases exit from the bag. The gas outlet may simply be a hole in the wall of the reservoir bag, through which exhaled gas may enter the collecting tube, or, it may take the form of a structure such as a bag tail, of the sort conventional in reservoir bags of breathing systems. The use of a gas outlet in the form of a bag tail may be preferred, since these structures are familiar to operators. However, it will be appreciated that in the breathing systems of the invention the gas outlet communicates with the collecting tube (in contrast to conventional closed bag tails) without allowing waste gas to enter the operating room environment (in contrast with conventional open bag tails).

The reservoir bag and tube may be arranged such that the direction of flow of gas in the collecting tube is both substantially parallel to, and substantially opposite to, the direction of flow of gas between the gas inlet and gas outlet of the bag (i.e. so that the first and second directions of gas flow are substantially counter to one another). This arrangement provides a number of advantages to the breathing systems of the invention. Since exhaled gas flowing in the collecting tube is directed back towards the patient the requirement for further tubes or conduits to extend substantially beyond the reservoir bag is removed. Accordingly the breathing system of the invention is less subject to entanglement, kinking or snagging during use. This improves the convenience of use of the breathing system, and renders it less cumbersome to an operator. Furthermore, this arrangement increases the extent to which the weight of a conduit carrying exhaled gas from the breathing system to a gas collector (which may, for example, be part of an anaesthetic machine) may be borne by an operator holding the patient adaptor (such as a face mask). This helps to reduce the tendency of the weight of the conduit leading to the gas collector to distort or obstruct the breathing system. An arrangement in which the first and second directions of gas flow are substantially counter to one another may be obtained by folding a reservoir bag and collecting tube, so that the reservoir bag and collecting tube are laterally offset from one another.

For the purposes of the present disclosure, it should be recognised that a collecting tube for use in a breathing system in accordance with the present invention need not be of cylindrical form, and may have any suitable cross sectional shape. Suitable embodiments may have an approximately square, “D”-shaped or rectangular cross section.

The extension of at least part of the walls of the bag beyond the periphery of the collecting tube aids an operator's ability to observe expansion and contraction of the bag caused by a patient's breathing. The ability of an operator to check a patient's breathing by reference to expansion and contraction of the reservoir bag is of great benefit in monitoring the patient's health during anaesthesia. Previous attempts to overcome the failings of the prior art have suffered from the fact that the reservoir bag may be obscured from the operator, thus meaning that the ability of the operator to visually inspect the integrity of the bag (for example to confirm that there are no tears or holes present) is impaired. Furthermore, a valuable aid in the monitoring of a patient's breathing is lost. It may generally be preferred that the collecting tube is of a size such that it does not substantially obscure the visibility of the reservoir bag. This may, for example, be achieved by use of a collecting tube having a cross section that is smaller than the cross section of the reservoir bag.

The volume of a reservoir bag for use in a breathing system in accordance with the invention may be in the region of between about 500 ml and 1,000 ml. It may be preferred that the collecting tube has a volume that is smaller than the volume of the reservoir bag. The use of bags and collecting tubes having relatively small volume is of benefit in that it reduces compression volumes associated with the breathing system.

The breathing system of the invention may preferably comprise a T-piece. T-piece breathing systems allow an operator to “feel” the compliance of a patient's lungs, with compliance being reflected in resistance of the reservoir bag to compression. Previous modifications of T-piece breathing systems designed to reduce atmospheric pollution by exhaled gases have suffered from the disadvantage that this feeling is lost, for instance in modifications in which the reservoir bag is enclosed. Breathing systems in accordance with the present invention, in which the reservoir bag is covered by the collecting tube over only a relatively small part of its surface, maintain the benefit of being able to feel compliance of the lungs. The flexibility of the bag may also of benefit in preserving an operator's feeling for the compliance of a patient's lungs. The inventors have found that a breathing device utilising a reservoir bag and collecting tube manufactured from polychloroprene and with walls approximately 0.3 mm thick is well suited to provide an operator with useful information regarding the compliance of a patient's lungs.

In a breathing system in accordance with the present invention, the collecting tube is attached to an outer surface of the reservoir bag. The collecting tube may preferably be attached to the outer surface of the reservoir bag along the majority of its length, and more preferably along all (or substantially all) of its length. The collecting tube is preferably permanently bonded to the outer surface of the reservoir bag. A suitable permanent bond may be achieved using an appropriate adhesive. The attachment of the collecting tube to the outer surface of the bag provides a number of advantages. Attachment of the collecting tube to an outer surface of the bag may help to reduce the potential for the collecting tube to become snagged in use. Furthermore, attachment of the collecting tube to an outer surface of the reservoir bag makes the device easier to clean. A further advantage lies in a reduction of the potential for the collecting tube to kink, and thereby become occluded leading to a build up of gas pressure. This is reduced since the ability of the collecting tube to move relative to the bag is removed or reduced. The likelihood of kinks forming in the collecting tube may also be decreased by integral formation of the reservoir bag and collecting tube.

In a preferred embodiment of a breathing system in accordance with the invention, the collecting tube is formed integrally with the reservoir bag. The collecting tube may be an extension of the gas outlet. The collecting tube and bag may be formed by a moulding process, such as dip moulding. The skilled person will appreciate that any collecting tube may be used as long as it is able to communicate with the gas outlet, and in use to connect to a gas collector. The use of a collecting tube that is formed integrally with the reservoir bag offers significant advantages over breathing systems in which the collecting tube and bag are formed separately. Merely by way of example, the ability to form both the bag and collecting tube in the same dip moulding technique reduces the number of steps in the manufacturing process, allowing the cost of production to be reduced. The use of breathing system employing integrally formed reservoir bag and collecting tubes also provides a number of advantages in terms of the function of the system, and some of these advantages are considered below.

A breathing system in which the bag and collecting tube are formed and supplied separately must be assembled prior to use. It will be appreciated that this assembly may be time consuming for those undertaking it. This disadvantage is obviated by the use of a breathing system in which the reservoir bag and collecting tube are formed integrally. Furthermore, there is a risk that the components may be incorrectly assembled, thus impairing the function of the breathing system. Incorrect assembly of the components may cause blockage of the flow of gasses through the bag and collection tube. If waste gases are not able to properly exit the reservoir bag, then pressure can build up within the bag. This can lead to the risk of hyperbaric injury to the patient, or to accidental introduction of gases into the patient's stomach.

Even if correctly assembled, the function of a breathing system incorporating a separately formed reservoir bag and collecting tube may be impaired if the connection between these elements loosens or fails during use. Incorrect assembly or loosening of the tube and bag may lead to unintentional disconnection of these components. Such disconnections may be partial or total, and may cause a number of unwanted effects. In particular, leakage of exhaled gasses from the breathing system may cause release of anaesthetic gasses into the operating room, and may thus significantly detract from the intended function of the system.

The benefits provided by the use of an integrally formed collecting tube and reservoir bag appear to outweigh any potential advantages that may be suggested to arise from the ability to detach the collecting tube and bag from one another and thus disassemble or convert the system during use (for example by disconnecting the system from the gas collector). There is an advantage in providing a breathing system that reduces the likelihood of occlusions occurring, and which maintains the ability to safely transport waste gases to a gas collector. If a breathing system is disconnected from a scavenger in the event of an occlusion occurring, this will allow potentially harmful waste gases to be released into an operating room, thus negating the benefits that the system seeks to provide.

It will be appreciated that when the collecting tube is formed as an extension of the gas outlet communication between the two may arise as a result of their continuity of formation. When the collecting tube and reservoir bag are formed separately, the gas outlet may communicate with the tube by means of any suitable connection. In the case that the reservoir bag and collecting tube are formed separately, it may be preferred that the reservoir bag and collecting tube are permanently connected to one another, in order to avoid many of the potential disadvantages outlined above.

It may be preferred that the gas outlet communicates with the collecting tube via an occludable, or partially occludable, aperture. Such an aperture may preferably be occludable by external application of pressure by an operator. The ability to occlude, or partially occlude, the communication between the gas outlet and collecting tube is of benefit when the breathing system is to be used to ventilate a patient. Breathing systems may be used to ventilate patients when spontaneous breathing fails. Pressure exerted by the operator on the reservoir bag allows gas to be returned to the patient's lungs. It is conventional that reservoir bags for use in prior art T-piece breathing systems are partially occluded by compressing the gas outlet of the bag. This reduces the flow of gases through the open bag tail and promotes the return of gases from the reservoir bag to the patient. It is preferred that reservoir bags for use in the breathing systems of the invention may also be occluded or partially occluded in this manner, since this will increase familiarity of the novel breathing systems to an operator.

A preferred example of an occludable aperture in accordance with this embodiment of the invention may be a hole between an extension of the gas outlet and a wall of the collecting tube, via which the collecting tube and gas outlet communicate. Alternatively, a suitable hole may be between a portion of the reservoir bag wall proximal to the gas outlet and a wall of the collecting tube, allowing communication between the reservoir bag and collecting tube. Suitable apertures may allow gas to enter the collecting tube substantially laterally to the first and/or second directions of gas flow.

A suitable hole may have a diameter in the region of 0.5 cm to 1.5 cm. In the case that the hole is formed in an extension of the gas outlet it may be preferred that the extension is sufficiently broad that the hole, of whatever size is selected, does not extend fully across the extension.

An apparatus of the invention may employ a single hole or a plurality of holes communicating between the collecting tube and the bag wall or wall of the gas outlet. A plurality of holes, for instance two or three holes, may be used to facilitate the flow of gasses. As described in the Experimental Results section below, the inventor has found that increasing the number of apertures by which gas may enter the collecting tube serves to decrease resistance in the breathing system.

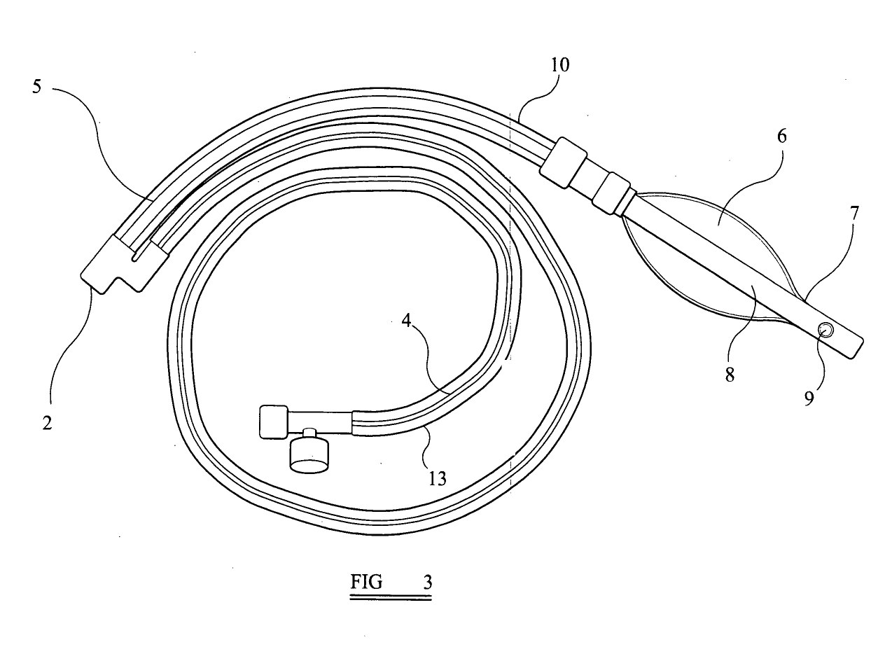

In any of the embodiments described above, such a hole may be the sole route (or in the case of embodiments with a plurality of holes, routes) by which the gas outlet and the collecting tube communicate, or may be present along with other passageways by which the reservoir bag and collecting tube may communicate. For example, the reservoir bag and collecting tube may communicate by a passageway produced by the extension of the gas outlet to form the collecting tube. In apparatuses of the invention incorporating holes of the sort described above, a proportion of gas flow may still take place via the other passageways provided, even if these are “downstream” of the holes (i.e. located such that exhaled gas has to pass the holes in order to enter the alternative passageway). The proportion of gasses flowing via the various different routes will alter depending on their relative resistances to the passage of gas. The inventor has found that in the embodiment shown in FIG. 2 below, the resistance to passage of gas via the folded end of the bag extension, is relatively high and that most gas passes from the extension of the gas outlet to the collecting tube via the hole provided.

When the occludable aperture comprises a hole as described above, the hole may be occluded, or partially occluded, by pressure on the external surface of the gas outlet, bag wall or collecting tube sufficient to substantially prevent gas flow through the hole. In the event that the hole is provided in addition to a further passageway by which the reservoir bag and collecting tube may communicate, it may be preferred that the hole is positioned such that when pressure is applied to occlude (or partially occlude) the hole, this will also cause occlusion (or partial occlusion) of the other passageway. In the example described in more detail below, an arrangement in which occlusion of the aperture also serves to occlude gas flow in a further passageway linking the reservoir bag and collecting tube is achieved by use of an aperture that connects a section of the gas outlet “upstream” of the further passageway (with reference to the direction of gas flow in normal use) with a section of the collecting tube “downstream” of the further passageway. Thus application of pressure to occlude the aperture also causes the further passageway to be effectively sealed.

It is conventional for reservoir bags of breathing systems to be provided with means to prevent their occlusion. In the case of breathing systems in accordance with the invention, the collecting tube may advantageously be provided with means to prevent its occlusion. Suitable means to prevent occlusion may be provided inside the collecting tube. Examples of suitable means that may be employed in a breathing system in accordance with the invention include pliable stents, such as open cell sponges, rigid tubes (or arrays of tubes) in which gases may flow through or between the tubes, and corrugated formations (in which gases may flow between the corrugations). Anti-occlusion means of this sort may be of benefit in keeping the passageways open thus aiding a patient's breathing and preventing the development of dangerous high pressures within the system. Anti-occlusion means, such as pliable stents, may also serve to reduce vibration that may otherwise occur in the flexible materials of a reservoir bag or collecting tube. Such vibration is a cause of resistance, and thus reducing vibration serves to reduce resistance in the breathing system.

In the case of breathing systems of the invention which incorporate an occludable, or partially occludable, aperture (as described above), it may be preferred that any means to prevent occlusion are omitted from the region of occludable aperture, or that the anti-occlusion means are able be overridden by the application of external pressure to occlude the aperture. Yeates drain, and in particular three strand Yeates drain, has been found to represent an especially preferred example of a pliable stent for provision in the collecting tube, since the inventor has found that it provides little resistance to the flow of gases in the collecting tube, but provides significant protection against inadvertent occlusion.

Turning now to the breathing conduits, a first conduit leading to a face mask may communicate with the exhaled gas inlet, a second conduit leading to a gas collector may communicate with the collecting tube, and a third conduit leading to the face mask may communicate with a fresh gas source. The second conduit may comprise first and second portions (as illustrated in the accompanying drawings), the first portion extending from the collecting tube to the face mask and optionally a T-piece, the second portion extending from the face mask and/or T-piece to the gas collector. The first conduit may be bundled together with a first portion of the second conduit. The first conduit may be enclosed within the first portion of the second conduit to form a coaxial tube.

Preferably the first and third conduits meet at a common junction in the form of a T-piece. The third conduit may be bundled together with the second portion of the second conduit. The third conduit may be enclosed within the second portion of the second conduit to form a coaxial tube. The first and second portions of the second conduit may also meet at a common junction formed by the T-piece, which may be at a point close to the patient's mouth. Positioning of the T-piece close to the patient's mouth, and removed from the reservoir bag, reduces the tendency for the weight of the T-piece to drag at the bag. Such drag may cause kinking around the bag's exhaled gas inlet, which may impair the function of the breathing system.

In order to reduce resistance to gas flow within an outer conduit of a coaxial tube the inner conduit may be markedly smaller than the outer conduit in which it is enclosed. The inventor has found that the third conduit leading to the fresh gas supply may be formed from pressure tubing with a cross section in the region of 3.5 mm and still serve to provide sufficient fresh gas to a patient at pressure that will be tolerated by the anaesthetic machine without causing activation of associated alarms. The use of pressure tubing with a relatively narrow cross section means that the space taken up by the third conduit within the second conduit is reduced. It also means that, in embodiments where the third conduit is bundled within the second conduit to form a coaxial tube, the size of the tubing from which the outer conduit is formed can be reduced. The inventor has found that, if the third conduit is formed from pressure tubing with a cross-section in the region of 3.5 mm, then the second conduit may be formed from tubing with a cross-section in the region of 15 mm. This significantly reduces the overall bulk of the breathing system.

The inventor has also found that, when the second conduit (and in particular the second part of the second conduit) comprises reinforced smooth bore tubing this is able to markedly decrease resistance in the breathing systems of the invention. More information regarding this embodiment is provided in the Experimental Results section below.

Although preceding paragraphs refer to bundled arrangements of the conduits in which coaxial tubes are formed it will be appreciated that suitable bundled arrangements are not limited to coaxial tubes, but may also include use of parallel tubes (such as abutting D-shaped tubes) and split-lumen tubes (for example, in which septum divides a tube in a manner to provide more than one conduit).

The nature of a breathing system in accordance with the present invention will be familiar to medical staff, removing the need for training in its use and reducing the likelihood of mistakes. The system's construction is such that a medical practitioner can easily institute hand ventilation while monitoring the patient's airway and respiratory movements. Positioning of an occludable aperture between an extension of the gas outlet and the collecting tube helps to increase the familiarity of the device, since occlusion is achieved by pinching the region of the gas outlet in the same manner as with previously known devices. A breathing system in accordance with the invention may preferably be a “two handed system” (in which a single operator is able to attend to ventilation of a patient using the reservoir bag and also to maintain suitable contact between the patient adaptor and the patient's face).

The breathing system can be constructed reasonably cheaply and may be disposable, thus reducing the risks of cross-infection between patients. While particularly suitable for paediatric anaesthetics the breathing system can also be used in adult anaesthetics, in intensive care units and other applications in which it is wished to ventilate human patients, and in veterinary work.

Embodiments of the present invention will now be described, by way of example, with reference to the accompanying drawings, in which:

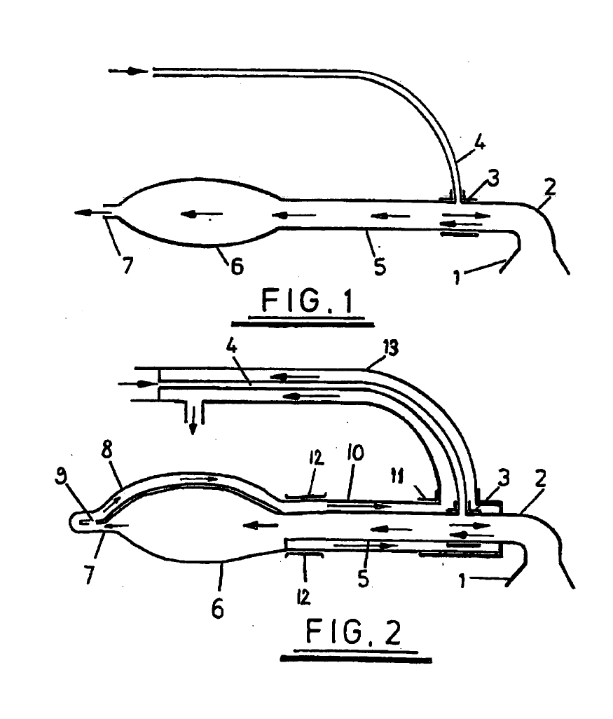

FIG. 1 is a schematic representation of a prior art device;

FIG. 2 is a schematic representation of a breathing system in accordance with the present invention;

FIG. 3 is a representation of the breathing system schematically illustrated in FIG. 2;

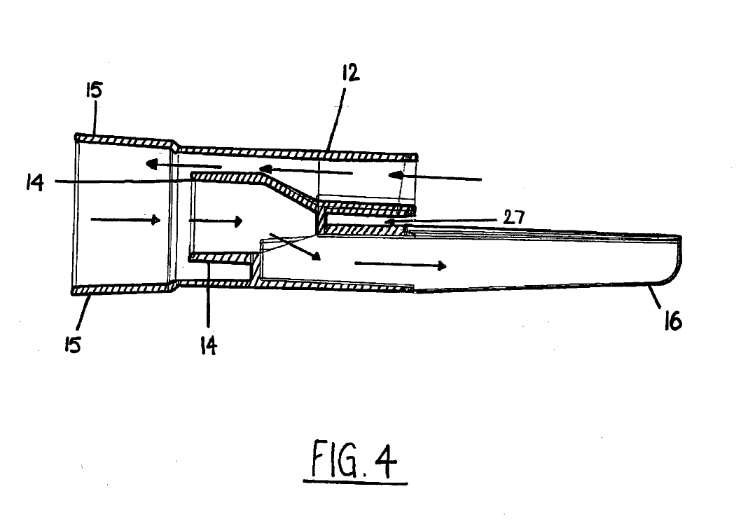

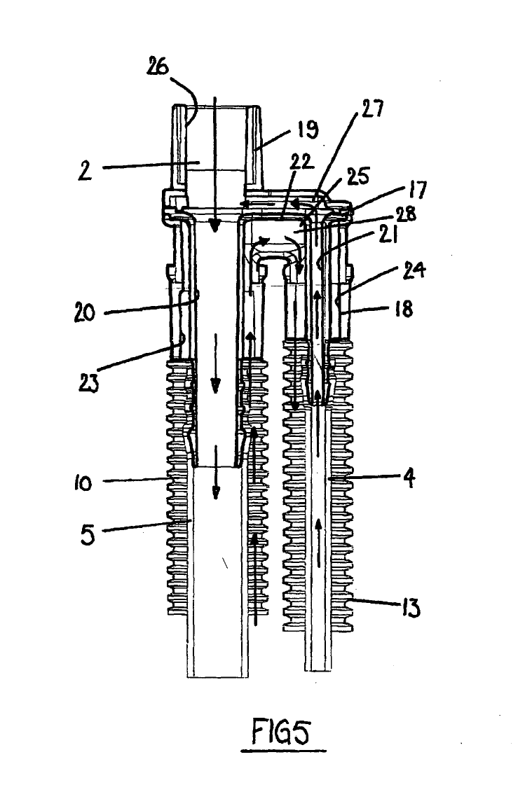

FIG. 4 is a section through a connector that may be used to connect the reservoir bag and collecting tube of a breathing system of the invention to a conduit;

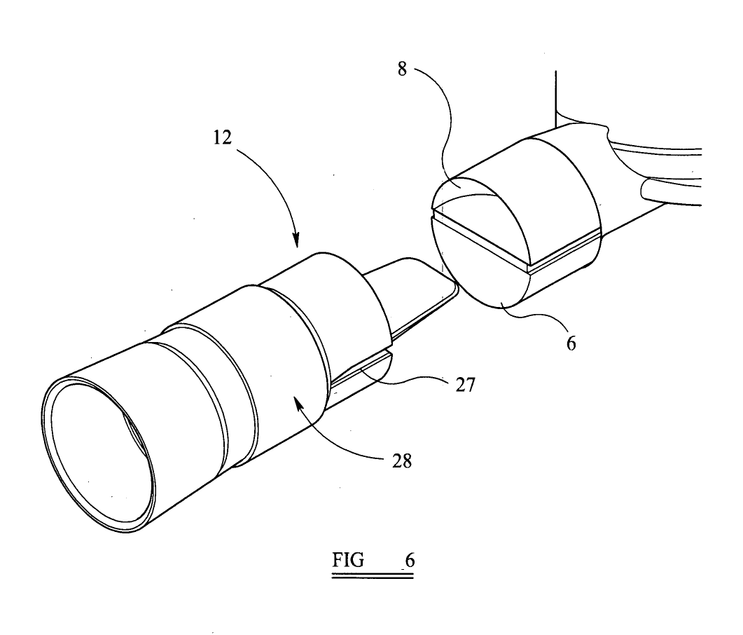

FIG. 5 is a section through a coaxial T-piece used in the breathing system of FIG. 3;

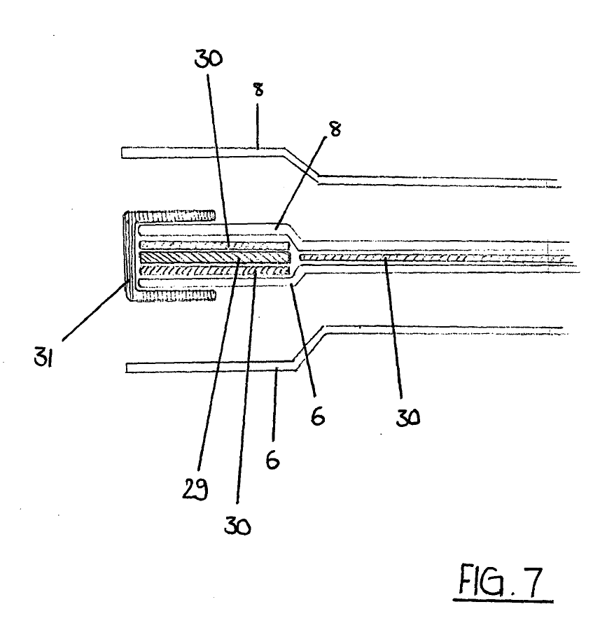

FIG. 6 illustrates a preferred means by which the connector, bag and collecting tube may be connected to one another;

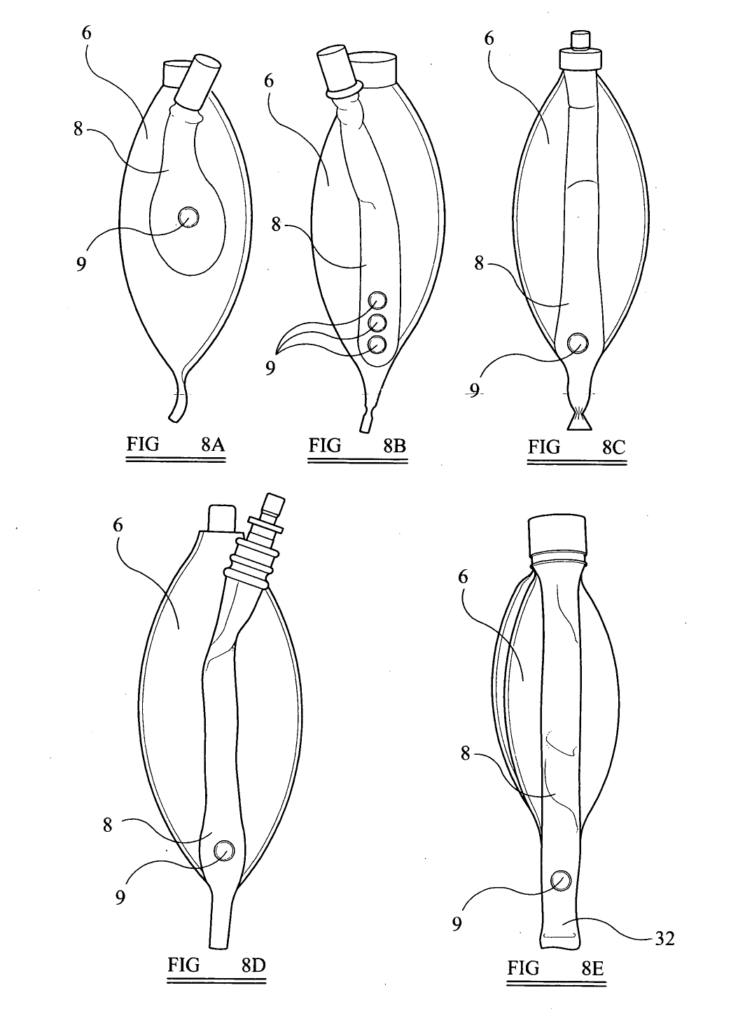

FIG. 7 is a section through the site at which the gas inlet of the reservoir bag and the collecting tube are attached to one another;

FIG. 8 is a representation illustrating arrangements of reservoir bags and collecting tubes in a number of embodiments of breathing systems in accordance with the present invention; and

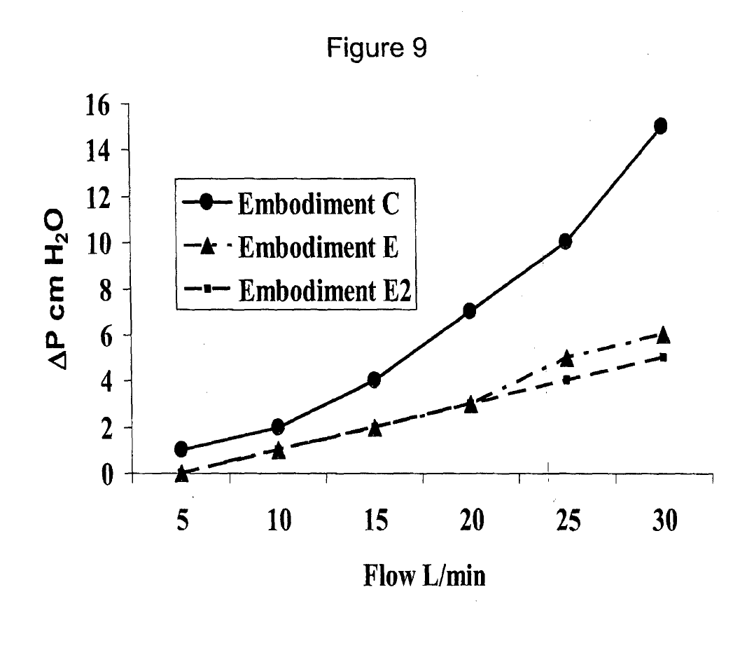

FIGS. 9, 10 and 11 are graphs, referred to in more detail in the Experimental Results section, that compare pressure across various breathing systems or breathing system components, in response to varying rates of gas flow.

In FIGS. 1 and 2 arrows are used to denote the direction of flow of gas through the apparatus. Where appropriate, the same reference numerals are used in all the figures for equivalent components.

Referring to FIG. 1, in the illustrated conventional (Jackson Rees T-piece) breathing system, a face mask 1 which may be positioned over a patient's nose and mouth is connected to a conduit 2 through which fresh and exhaled gases flow. A T-piece 3 connects conduit 2 to a conduit 4 having its other end attached to a fresh gas source. The third limb of the T-piece connects to a conduit 5 which has its other end attached to a reservoir bag 6. Exhaled gas leaves the reservoir bag through an open tail defining a gas outlet 7. In use, the bag 6 expands as the patient exhales, and contracts somewhat as the patient inhales.

Referring to FIG. 2, in the illustrated embodiment of the invention the reservoir. bag 6 does not have an open tail, but instead the bag's gas outlet 7 is extended to define a collecting tube 8. Collecting tube 8 is folded so that it lies over the surface of the bag 6, and is attached to the surface of the bag by means of a permanent bond (the gap shown in FIG. 2 between the collecting tube 8 and bag 6 is only for illustrative purposes, and is not actually present). The gas outlet 7 and collecting tube 8 are able to communicate as a result of their continuity of formation, but in practice do so mainly via a hole 9 formed through a wall of the gas outlet and a wall of the tube. Tube 8 is connected to a conduit 10 that carries exhaled gases from the bag 6 to an outer T-piece 11 which encloses the inner T-piece 3, such that these inner and outer T-pieces together define a coaxial T-piece. Conduit 10 encloses conduit 5 to form a coaxial tube where conduit 5 is of smaller diameter. The end of the bag 6 though which gas enters is fixed in position relative to the collecting tube 8 as a result of its connection to conduit 5 and the connection of collecting tube 8 to the conduit 10. The substantially parallel flows of gas from conduit 5 into the bag 6, and from collecting tube 8 into conduit 10, are converted into coaxial flows by a connector 12. The T-piece 11 has one limb terminated and a third limb connected to a conduit 13 which carries exhaled gas to a scavenging system (not shown). Conduit 13 encloses conduit 4 to form a second coaxial tube.

Referring to FIG. 3, in the illustrated breathing system the conduit 2 is in the form of a standard adapter which may be used to make a connection to a standard mask (not shown). Fresh gas travels along conduit 4 which is enclosed within conduit 13. Exhaled gas is passed along conduit 5 to the reservoir bag 6. Exhaled air leaves the reservoir bag via the bag's gas outlet 7, and enters the collecting tube 8, mainly via hole 9. From the collecting tube 8, exhaled gas travels through conduit 10, passes through the outer T-piece and passes along the conduit 13 to a scavenging system (not shown). Conduit 5 is enclosed within conduit 10 to form a coaxial tube.

The flexible walls of the reservoir bag 6 and collecting tube 8 allows the communicating region between the bag and tube, including hole 9, to be sealed on application of pressure by the medical practitioner's hand. This allows ventilation of the patient if necessary. When the breathing system is used to ventilate a patient, pressure applied through the walls of reservoir bag 6 causes gas to be expelled towards the patient along conduit 5, thereby inflating the patient's lungs.

FIG. 4 shows a connector 12 that may be used to connect the reservoir bag 6 and collecting tube 8 to a coaxial tube comprising conduits 5 and 10. An inner part 14 of the connector 12 engages tightly with the conduit 5, and directs gases within this conduit into the exhaled gas inlet of reservoir bag 6. An outer part 15 of the connector 12 engages with the collecting tube 8 and conduit 10, and thereby directs exhaled gas from the collecting tube into the conduit (which in this case constitutes the outer portion of the coaxial tube). The tight connection of inner part 14 with conduit 5 ensures that the flow of gases in the conduits 5 and 10 is kept separate. A rounded blade 16 extending from the connector 12 into the reservoir bag 6 aids in the maintenance of an open passageway through which gases may flow. A slot 27 between the blade and the upper part of the connector 12 is provided to receive a region at which the inhaled gas inlet of the bag 6 and the collecting tube 8 are connected. Assembly of the connector 12, bag 6 and collecting tube 8 is described in more detail in FIGS. 6 and 7 below.

FIG. 5 shows a coaxial T-piece comprising the inner and outer T-pieces 11 and 3 which interconnect conduits 2, 4 and 5 and 10 and 13. The coaxial T-piece is formed from three parts, an inner portion 17, an outer portion 18, and a cap portion 19. Inner portion 17 comprises substantially parallel cylindrical bores 20 and 21, linked by an inner bridging part 22. These are located within corresponding parallel cylindrical bores 23 and 24, and a corresponding outer bridging part 25, of the outer portion 18, such that bores 20 and 23 are coaxial, as are bores 21 and 24. The cap portion 19 defines a further cylindrical bore 26, and the cap portion attaches to inner portion 17 to provide a gas tight seal.

The inner T-piece 3 is defined by the cap 19 and the inner portion 17. Bore 20 defines a first limb of the T-piece, which is connected to conduit 5. Bore 26 of the cap portion 19 defines a second limb of the T-piece which may be attached to a face mask using a standard adapter 2 shown in FIGS. 3 and 4 but not shown in FIG. 6. A space 27 defined between the cap portion 19 and the inner portion 17 provides a third limb of the T-piece. Connection of this third limb to conduit 4 via bore 21 allows passage of fresh gas to the face mask through the second and third limbs of the T-piece.

The outer T-piece 11 is defined by the inner portion 17 and outer portion 18, which connect to one another to form a gas tight seal. The space defined between cylindrical bores 20 and 23 provides a first limb of the outer T-piece 11, while space 28 between the inner and outer bridging portions 22 and 25 provides a second limb. The third limb of the outer T-piece 11 is sealed by gas tight connection between the inner portion 17 and outer portion 18.

A face mask may be attached to the inner T-piece 3 via bore 26, which is sized to receive a suitable attachment. Gases flow to the patient along conduit 4, pass through the inner T-piece 3, and through bore 26 to the face mask. Exhaled gases pass into the inner T-piece through bore 26, pass through the inner T-piece 3, and pass through conduit 5 to the reservoir bag 6. Exhaled gases returning from the collecting tube 8 pass along conduit 10 into the outer T-piece 11 and leave to a gas collector through conduit 13.

FIG. 6 illustrates a preferred manner in which a connector 12 may be attached to a bag 6 and collecting tube 8. An elastic band 28 is placed around the connector 12, which has the same form as described in FIG. 4. The connector 12 is then inserted into the bag 6 so that rounded blade 16 enters the exhaled gas inlet of the bag, and so that a site at which the exhaled gas inlet of the bag and the end of collecting tube 8 are attached to one another is received in slot 27. Once the connector 12, bag 6 and collecting tube 8 have been engaged tightly together the elastic band 28 is rolled over the region at which the connector, bag and collecting tube engage, thus keeping these elements in place.

The attachment of the exhaled gas inlet of bag 6 and the end of the collecting tube 8 is illustrated schematically in cross-section in FIG. 7. Here it can be seen that this region is stiffened by the presence of a die cut sheet 29 of plastic 1 mm thick. This is attached to both the exhaled gas inlet of bag 6 and the collecting tube 8 by adhesive 30, which thus attaches the bag and tube to one another. The adhesive 30 may be the same adhesive used to attach the collecting tube to the outer surface of the bag elsewhere. A layer 31 of single sided self adhesive low friction tape is folded over the stiffening sheet 29 and adhesive 30 and affixed to an inner surface of the exhaled gas inlet of the bag 6 and an inner surface of the collecting tube 8, such that the tape substantially covers the sheet and adhesive. This layer 31 serves to further stiffen the region by which the bag 6 and collecting tube 8 are attached, and also aids in the smooth attachment of the bag and tube to the connector 12.

Turning now to FIG. 8, five embodiments of a breathing system of the invention are illustrated, respectively labelled A to E. Each comprises a reservoir bag 6 and collecting tube 8. As shown, embodiments A to D are adapted to connect to a parallel, rather than coaxial, arrangement of conduits leading to the to the patient and gas collector; however, with a suitable connector they could be so attached. D utilises a reservoir bag 6 with a volume of 1000 ml.

In each of the embodiments A to E the reservoir bag 6 and collecting tube 8 communicate with one another via holes 9. Embodiment B is provided with three communicating holes 9. In embodiments A to D the holes 9 represent the sole passageway via which gases may pass from the reservoir bag 6 into the collecting tube 8. In Embodiment E, which corresponds the breathing system illustrated in FIG. 2, the hole 9 is the main route by which gas may pass from the reservoir bag 6 into the collecting tube 8. It can be seen that in embodiment E an extension of the reservoir bag's gas outlet 7 has the form of a broad bag tail 32. The gas may also pass from the gas outlet 7 to the collecting tube 8 via this tail 32, as well as through hole 9. The tail 32 is wider than the diameter of hole 9, and it may be preferred that the tail is relatively short to reduce the path travelled by the gas.

In each of embodiments B to E, the hole (or holes) 9 are located such that they may be occluded or partially occluded by the operator in a manner conventional in the prior art.

### EXPERIMENTAL RESULTS

The inventor investigated the effect on pressure levels across a breathing system of the invention of varying the number of passages by which gases enter the collecting tube. The results of this investigation are shown in accompanying FIGS. 9 to 11.

Each of FIGS. 9 to 11 takes the form of a graph plotting the change in pressure (ΔP shown on the Y-axis calibrated in units of centimetres of water—cm H₂O) with gas flow through the breathing system or breathing system components (shown on the X-axis calibrated in litres of gas per minute—L/min). The higher the value on the Y-axis, the greater the resistance to gas flow within the breathing system or breathing system component.

FIG. 9 compares the resistance to gas flow occurring in various embodiments of a breathing system of the invention. The embodiments investigated correspond to embodiment C and E of FIG. 8, and a further embodiment (referred to as E2) similar to embodiment E, but provided with two holes formed between the extended gas outlet and collecting tube.

The system of embodiment C is an early prototype constructed from available materials; in contrast embodiments E and E2 which are constructed from specially designed and produced parts.

The results illustrate that the embodiment C, exhibits higher resistance than embodiment E and E2. This probably reflects the much narrower collecting tube of embodiment C compared with those of E and E2, which were purpose built to be wider throughout their length. In addition, it is seen that the two-hole device E2 has somewhat less resistance at high flows compared with the one-hole version E. Without wishing to be bound by any hypothesis, the inventor believes that this decrease in resistance may be the result of decreasing the obstruction to the passage of gas from the bag to the collecting tube and/or shortening the pathway to be travelled by the gases when passing between these structures.

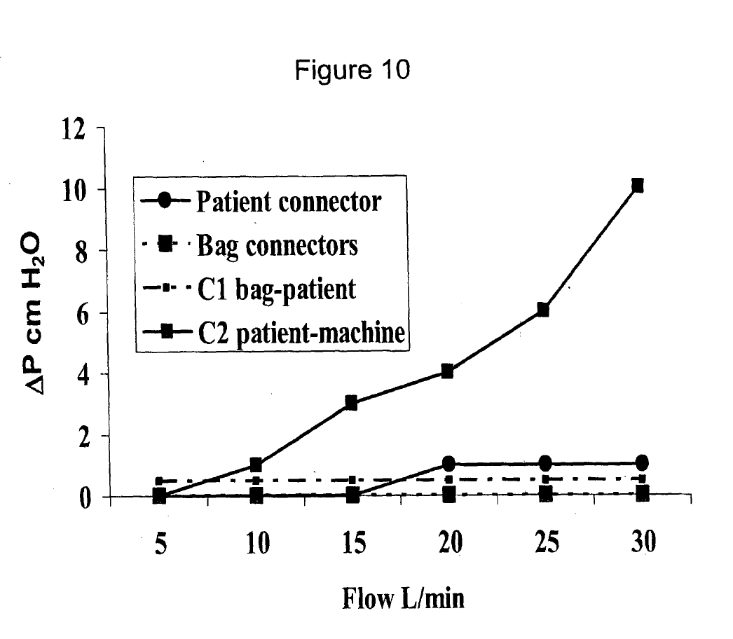

FIG. 10 compares the resistance of the connectors and tubes that comprise the gas collection system: i.e. the outer conduits of the coaxial tubes and connectors that run between the bag connector (connector 12 shown in FIG. 2) and the gas collection system situated on the anaesthetic machine (not shown). It is evident that the bag connector and the means by which it attaches to the tubes 5 and 10 (shown in FIG. 2), the outer conduit C1 (defined by tubes 5 and 10) that runs between the bag to the coaxial T-piece (shown in FIG. 2), and the outer conduit defined by the inner and outer portions of the coaxial T-piece (shown in FIG. 2 and FIG. 5) offer minimal resistance to gas flow (0-1 cm H₂O at 30 L/min gas flow). By contrast, the outer conduit C2, defined by tubes 4 and 13 that runs between the coaxial T-piece (shown in FIG. 2) and the gas collection device situated on the anaesthetic machine (not shown) has a resistance of 10 cm H₂O at 30 L/min and is therefore the main source of resistance in this part of the breathing system.

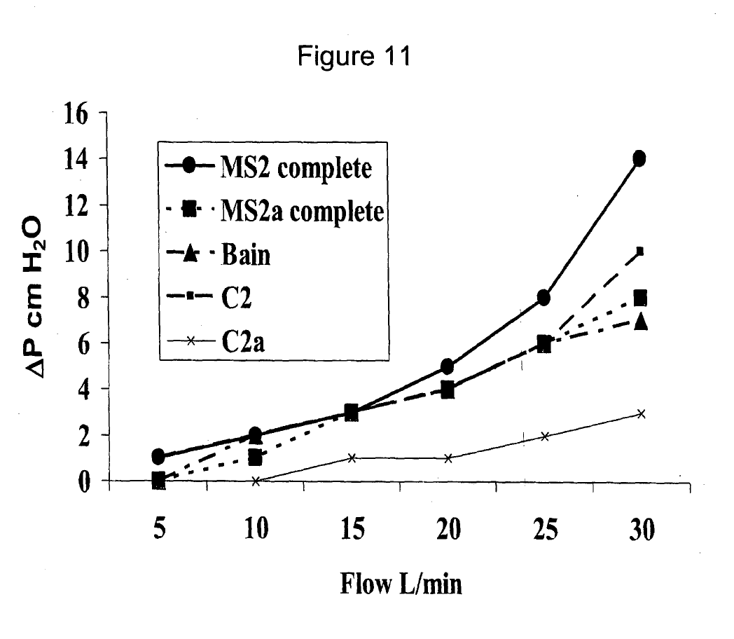

The inventor has found that substitution of the corrugated tubing of tube 13 (shown in FIG. 2) with reinforced smooth bore tubing significantly decreases the resistance associated with outer conduit C2, as shown in FIG. 11. Here, resistance was compared between a breathing system of the invention in which tube 13 was formed of corrugated tubing (designated “MS2 complete”) and a breathing system of the invention in which this tube was formed of 15 mm diameter reinforced smooth bore tubing (designated “MS2a”). It can bee seen that at a gas flow rate of 30 L/min the resistance in the breathing system in which tube 13 was formed of reinforced smooth bore tubing was approximately half that of the system in which tube 13 was formed of corrugated tubing (pressure drop of 14 cm H₂O across MS2 as opposed to 8 cm H₂O with MS2a). Comparison of resistance in the experimental conduits alone (C2 in which the outer tube was formed of 15 mm corrugated tubing and C2a in which the outer tube was formed of 15 mm reinforced smooth bore tubing) illustrated that the loss of resistance observed was entirely attributable to the use of reinforced smooth bore tubing (pressure drop 10 cm H₂O across C2 as opposed to 3 cm H₂O across C2a).

In light of the above, it may be preferred that the conduit that links the coaxial T-piece (patient connector as shown in FIG. 5) and the gas collection device situated on the anaesthetic machine in the breathing systems of the invention comprises reinforced smooth bore tubing, in order to decrease resistance to gas flow in the breathing system. Breathing systems of the invention in accordance with this embodiment exhibit resistance similar to prior art breathing systems that incorporate a valve to facilitate connection to a gas collector. This can be shown by comparing resistance in the “MS2” system and resistance in a standard Bain system (designated “Bain” in the graph of FIG. 11).

1. A breathing system comprising
a flexible reservoir bag having bag walls and provided with an exhaled gas inlet and a gas outlet, the bag defining a passageway for flow of gas in a first direction, and the gas outlet of the reservoir bag being in communication with a collecting tube which defines a passageway for flow of gas in a second direction and in use is connectable to a gas collector, wherein at least part of the walls of the bag extend beyond the sides of the collecting tube, and the first and second directions of gas flow are substantially parallel to, and laterally offset from, one another, and wherein the collecting tube is attached to an outer surface of the reservoir bag.

2. The breathing system according to claim 1, wherein the collecting tube is attached to the outer surface of the reservoir bag along the majority of its length.

3. The breathing system according to claim 1, wherein the collecting tube is permanently bonded to the outer surface of the reservoir bag.

4. The breathing system according to claim 1, wherein the collecting tube is formed integrally with the reservoir bag.

5. The breathing system according to claim 1, wherein the reservoir bag and collecting tube are arranged so that direction of flow of gas in the tube is substantially opposite to the direction of flow of gas between the gas inlet and gas outlet.

6. The breathing system according to claim 1, wherein the collecting tube has a cross section that is smaller than the cross section of the reservoir bag.

7. The breathing system according to any of claim 1, wherein the gas outlet communicates with the collecting tube via an occludable, or partially occludable, aperture.

8. The breathing system according to claim 7, wherein the occludable aperture comprises a hole between an extension of the gas outlet and a wall of the tube, via which the tube and gas outlet communicate.

9. The breathing system according to claim 7, wherein the reservoir bag and/or collecting tube may be provided with means to prevent their occlusion.

10. The breathing system according to claim 8, wherein the means to prevent occlusion is selected from the group consisting of: stents, open cell sponges, rigid tubes, and corrugated formations.

11. The breathing system according to claims 7, comprising a first conduit connected to a face mask communicating with the exhaled gas inlet of the inner reservoir bag, a second conduit connected to the exhaled gas outlet of the outer bag, and a third conduit which in use communicates with a fresh gas source.

12. The breathing system according to claim 11, wherein the first and third conduits are interconnected adjacent the face mask.

13. The breathing system according to claim 11, wherein the first conduit is bundled together with a first portion of the second conduit.

14. The breathing system according to claim 13, wherein the first conduit is enclosed within the first portion of the second conduit to form a coaxial tube.

15. The breathing system according to claim 13, wherein the third conduit is bundled together with a second portion of the second conduit.

16. The breathing system according to claim 15, wherein the third conduit is enclosed within the second portion of the second conduit to form a coaxial tube.

17. The breathing system according to claim 1, wherein the second conduit comprises smooth bore tubing.

18. The breathing system according to claim 17, wherein the second part of the second conduit comprises smooth bore tubing.

19. A breathing system substantially as hereinbefore described with reference to FIGS. 2 to 5 of the accompanying drawings.

20. The breathing system according to claim 9, wherein the means to prevent occlusion is selected from the group consisting of stents, open cell sponges, rigid tubes, and corrugated formations.

21. The breathing system according to claim 12, wherein the first conduit is bundled together with a first portion of the second conduit.

22. The breathing system according to claim 14, wherein the third conduit is bundled together with a second portion of the second conduit.
